(19) United States
(12) Patent Application Publication
(10) Pub. No.: US 2013/0321774 A1
(43) Pub. Date: Dec. 5, 2013

(54) Hand-Held Laser Device for Determining the Dominant Eye

(71) Applicants: Johan T.W. Van Dalen, Tucson, AZ (US); Dan D. Carda, Tucson, AZ (US)

(72) Inventors: Johan T.W. Van Dalen, Tucson, AZ (US); Dan D. Carda, Tucson, AZ (US)

(73) Assignee: EYE CARE AND CURE ASIA PTE. LTD., Singapore (SG)

(21) Appl. No.: 13/486,579

(22) Filed: Jun. 1, 2012

(51) Int. Cl. A61B 3/11; F21V 33/00; B32B 3/02

(52) U.S. Cl. 351/221; 428/80; 362/119

(57) ABSTRACT

A device for determining the dominant eye of a person. The device comprises a cone having a small hole at its apex which serves as a view port. The cone is attached to a handle, which contains a battery-powered, low-level laser light source that emits light through a light port in the handle. The cone and handle are designed to be made from a single sheet of material.

To determine a person's dominant eye, the person holds the device at arm's length by the handle. The laser is turned on and the person aims the light at a distant wall. The person looks through the base of the cone through the view port with both eyes open, and locates the laser spot on the distant wall through the view port. The person then closes his right eye. If the laser spot disappears from view, the person is right-eye dominant.

### FIELD OF INVENTION

This invention relates to instruments for determining a person's dominant eye. This invention relates particularly to a hand-held laser device for determining a person's dominant eye.

### BACKGROUND

Most people have a dominant eye, or one eye that looks directly at the object of regard. The non-dominant eye sees the object at a slight angle. Thus, when a person uses both eyes, the non-dominant eye is seeing a slightly different view. The difference between views gives humans depth perception.

It is important to know which eye is dominant for certain activities that require the person to judge the relative position of objects, such as sports that require accurate aim or making an accurate drawing of an object. It is also important to know which is eye is dominant for prescribing corrective eyeglasses and for corrective eye surgery. In surgical cases, where monovision (one eye for distance and one eye for close-up vision) is required, the dominant eye is usually set for distance vision and the non-dominant eye for close-up vision.

A common method for determining the dominant eye involves cutting a hole about one inch square in a piece of paper, holding it at arm's length, and focusing on an object through the hole with both eyes. The person then closes his right eye. If the object disappears from view, the person is right-eye dominant. If the person closes his left eye instead and the object disappears from view, the person is left-eye dominant. This method is simple, but is disadvantageous because it isn't standardized; for example the hole size, the object focused on, and the distance of the object focused on differs from person to person. It would be desirable to have a device that enables the eye dominance test to be standardized.

### SUMMARY OF THE INVENTION

This is a device for determining the dominant eye of a person. The device is a hand-held device, having a cone with a small hole at its apex which serves as a view port. The cone is attached to a handle, which contains a battery-powered, low-level laser light source that emits light through a light port in the handle. The cone and handle are designed to be made from a single sheet of material.

To determine a person's dominant eye, the person holds the device at arm's length by the handle, preferably by both hands at the person's nose level. The laser is turned on and the person aims the light at a distant wall. The person looks through the base of the cone through the view port with both eyes open, and locates the laser light spot on the distant wall through the view port. The person then closes his right eye. If the laser spot disappears from view, the person is right-eye dominant. Conversely, if the person closes his left eye and the laser spot disappears from view, the person is left-eye dominant.

### BRIEF DESCRIPTION OF THE DRAWINGS

FIG. 1 illustrates a front perspective view of an embodiment of the dominant eye device.

FIG. 2 illustrates a side view of an embodiment of the dominant eye device.

FIG. 3 illustrates a front view of an embodiment of the dominant eye device.

FIG. 4 illustrates a rear view of an embodiment of the dominant eye device.

FIG. 5 illustrates a perspective view of a light assembly.

FIG. 6 illustrates a front view of a light assembly.

FIG. 7 illustrates a cross-section view of the light assembly along line A-A of FIG. 6.

FIG. 8 is a plan view of one embodiment of a sheet to form the dominant eye device.

FIG. 9 is a plan view of a second embodiment of a sheet to form the dominant eye device.

FIG. 10 is a schematic illustration of a person using the dominant eye device (not to scale).

### DETAILED DESCRIPTION OF THE INVENTION

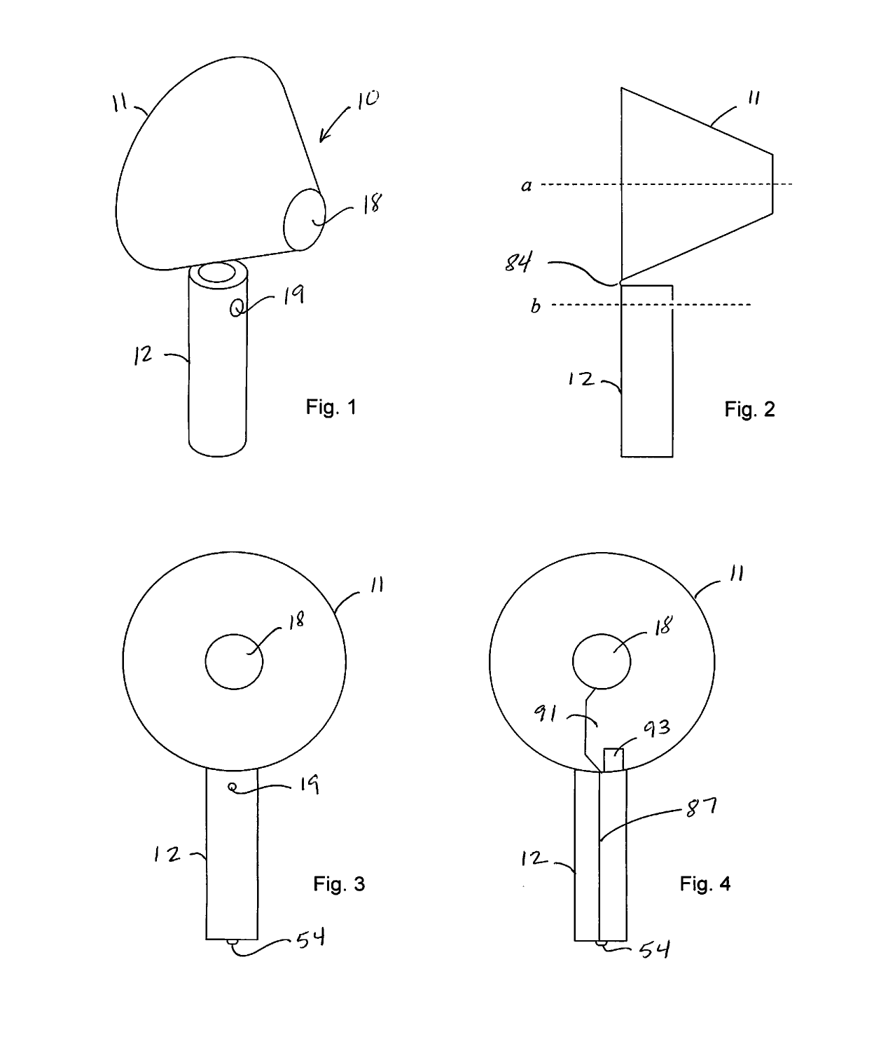

FIGS. 1-4 illustrate a device 10 for determining the dominant eye of a person. The device 10 comprises a cone 11 having a hole at its apex which serves as a view port 18. The cone 11 is attached to a handle 12, which contains light source that emits light such that the axis of the cone a is substantially parallel to the axis of the light b emitted from the device 10. Preferably the light is emitted through a hole in the handle which serves as a light port 19, but the light port may also be disposed in the cone or hinge.

Preferably the base of the cone 11 is larger than the person's interpupillary distance, so that the person looks through, not past, the cone when focusing on the light spot. Thus, because the average adult human interpupillary distance is considered to be between 60-70 mm, typically the base of the cone will be at least 7 cm in diameter. The view port 18 is preferably at least 12.5 mm and in the preferred embodiment is about 25 mm. A larger view port may suffice.

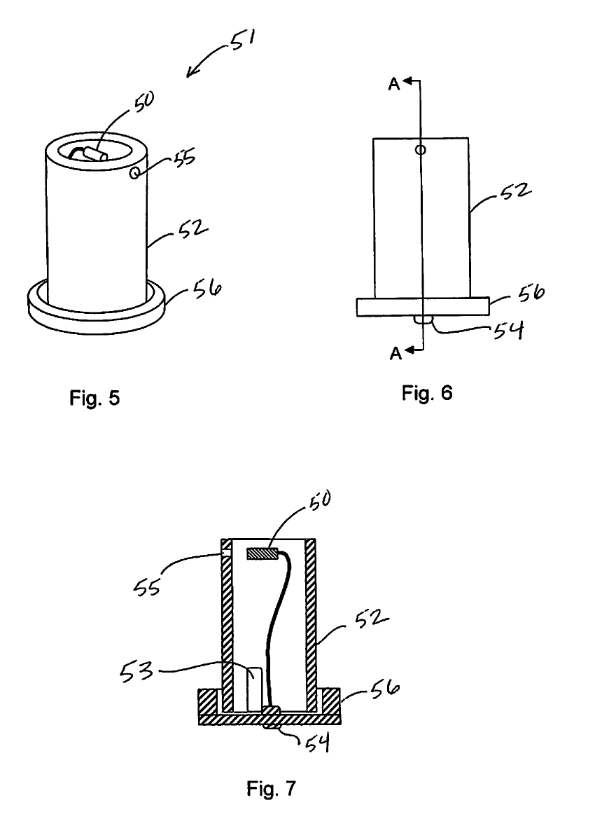

In a preferred embodiment, the light source is a battery-powered semiconductor laser diode 50, available commercially. A low-level laser diode is used to produce a tightly focused spot of laser light at a distance of about 5 m. The spot will preferably be about 2-3 mm in diameter. Preferably the laser diode emits red light. Also preferably the laser diode is small enough to fit inside the handle and emit light out the light port 19 without need for light-bending optical elements. That is, preferably laser diode is chosen that is short enough to be positioned along a diameter of the handle so that the axis of the light it emits is substantially parallel to a diameter of the handle. In a specific example, if the handle is perpendicular to the axis of the cone a, the diode is positioned so that the light it emits b is substantially perpendicular to the handle. Other light sources, such as incandescent lamps or LEDs may be suitable, although the heat and lack of focus at distance are disadvantageous relative to a laser light source. FIGS. 5-7 illustrate a light assembly 51, which comprises a housing 52 containing the laser diode 50, its battery 53, and a switch 54. The simplicity of the light assembly lends itself to hard-wired, discrete circuits, but printed circuit board, microchip, or a combination of these methods will suffice. The housing has an aperture 55 though which the light is emitted, which is aligned with the light port 19 to allow light to pass out of the handle 12. The light assembly 51 fits inside the handle 12, preferably snugly. The housing 52 is closed at one end to keep the components inside the housing 52. Preferably the housing 52 is closed with a cap 56 through which the switch 54 extends, so that the person can hold the handle 12 without touching the switch 54. Alternatively, the switch 54 may extend through the side of the housing 52 or the handle 12. The switch is preferably a momentary switch, but may also be a toggle switch.

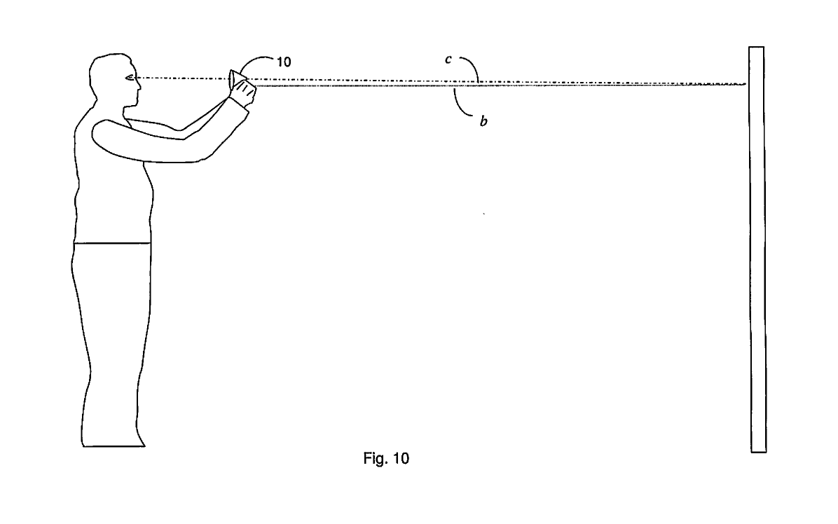

To determine a person's dominant eye, the person holds the device 10 at arm's length by the handle 12. See FIG. 10. Preferably the handle 12 is held with both hands at arm's length, and the base of the cone 11 should be at about the same height as the person's nose, referred to herein as the “nose level.” This to rule out interference of right- and left-handedness. Preferably, the axis of the cone a is to be held as, or parallel to, the person's line of sight c, so the axis of the emitted light b is also parallel to the line of sight. However, as long as the person can see the laser spot through the aperture in the cone, it isn't necessary for the line of sight to be parallel to the axis of the cone or to the axis of the emitted light. For example, if the wall is about 4.6 m away, there will be an area about 3.8 cm in diameter around the laser spot that can be seen through the view port 19, so perfect alignment between the axis of the cone a and the axis of the emitted light b is desirable, but not required.

At arm's length, view port 18 is held, on average, at about 84 cm from the person's eyes. The light source is turned on and the person shines the emitted light on a far wall. Preferably the distance from the person's eyes to the light spot on the far wall is about 4.6 m, which is easily attained in ophthalmologists' examination rooms, which are customarily at least that long. The person then looks through the view port 18 to locate the light spot on the distant wall. The person then closes his right eye. If the laser spot disappears from view, the person is right-eye dominant. Of course, if the person instead closes his left eye and the laser spot disappears from view, the person is left-eye dominant.

In the preferred embodiment, the patient holds the device 10 at arm's length while standing about 4.5 m from the wall. The patient looks through the 25 mm diameter opening at the apex of the conical section and locates the red laser spot on the wall with both eyes open. When the patient closes one eye, and the red dot disappears, the closed eye is the dominant eye.

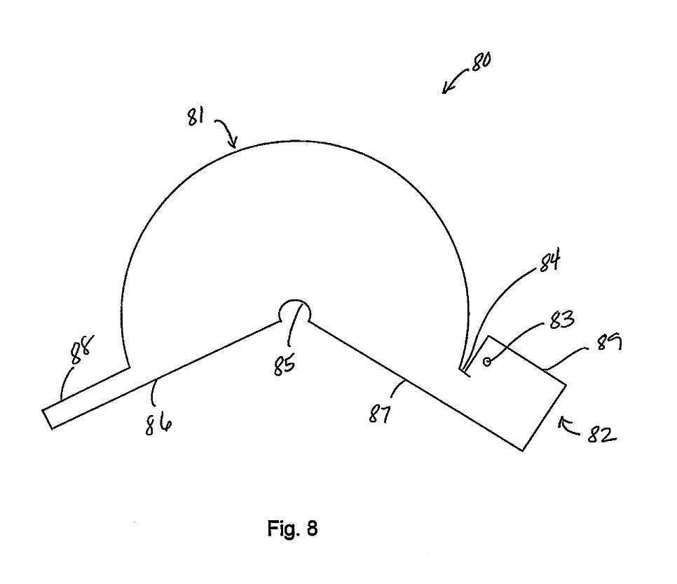

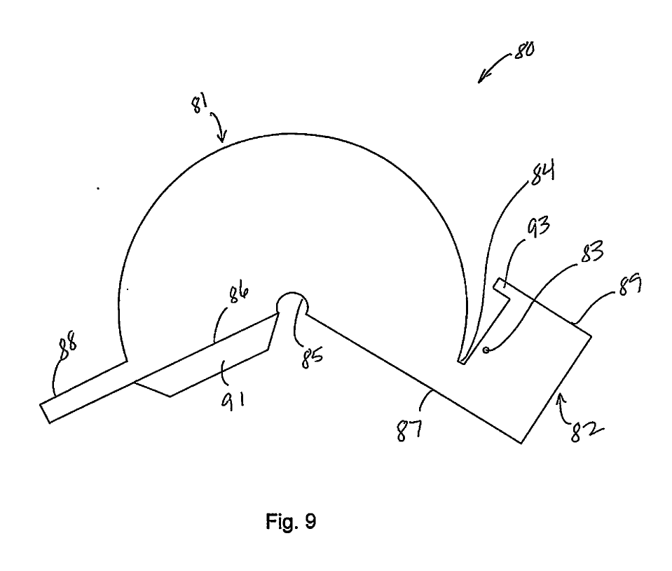

The cone 11 and handle 12 are preferably designed to be made from a single sheet of material. FIGS. 8 and 9 illustrate two embodiments of a flat sheet cut in a shape that can be formed into the present dominant eye device. The preferred embodiment is a sheet of 0.020″ opaque vinyl. Other embodiments can be made of any opaque, flexible material including thermoplastics such as ABS, polyethylene, polypropylene or other polymer. In the preferred embodiment, the sheets are cut using a laser. The edges are joined using a method appropriate for the material such as pop-riveting, fusion welding, ultrasonic welding, adhesive, etc. For example, edges of a vinyl sheet are bonded using conventional laser welding techniques.

As to FIG. 8, the sheet 80 is a single sheet of material having a portion 81 that is formed into a cone 11. The cone-formative portion 81 has a first edge 86 and a second edge 87 that are joined together to form the cone 11. Due to the shape of the sheet 80, a c-shaped edge forms a view port 18 when the first edge 86 and second edge 87 are joined together. Similarly, the single sheet of material has a handle-formative portion 82 that is formed into a handle 12. The handle-formative portion 82 has a third edge 88 and a fourth edge 89 that are joined together to form to form a cylinder, which becomes the handle 12. The handle-formative portion 82 also has an aperture 83 that serves as the light port 19. Once the cone 11 and handle 12 are formed, the device is bent along a hinge-forming portion 84 to position the cone 11 over the handle 12 such that the axis of the cone a is substantially parallel to the axis of the light b emitted from the light port 19. See FIG. 2.

FIG. 9 shows an alternative embodiment in which material extends from the first edge 86 to form a cone tab 91 that overlaps the second edge 87 to form the cone 11. FIG. 9 shows a tab 93 extending from the handle-formative portion 82 that overlaps and is connected to the base of the cone 11 near the intersection of the cone 11 and the handle 12. The handle tab 93 helps to keep the cone 11 in position over the handle 12 such that the axis of the cone a is substantially parallel to the axis of the light b emitted from the light port 19.

The single sheet manufacture enables lower-cost manufacturing than other methods. Alternatively, instead of a single sheet to manufacture the device, the device can be formed of a handle component attached to a cone component, such as a modified plastic funnel. The handle may be attached to a cone using a fastening means such as a weld, adhesive, or snap-fit plastic. Alternatively, the handle may be integral with the cone, for example as achieved by injection-mold manufacturing.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

We claim:

1. A device for determining the dominant eye of a person, the device comprising:
a) a cone having a hole at its apex which serves as a view port;
b) a handle attached to the cone, the handle having a light port; and
c) a light source contained in the handle which emits light through the light port.

2. The device of claim 1 wherein the handle is integral with the cone.

3. The device of claim 1 wherein the light source is a laser diode.

4. The device of claim 1 further comprising a battery connected to the light source.

5. The device of claim 1 wherein the diameter of the base of the cone is larger than the person's interpupillary distance.

6. The device of claim 1 wherein the light from the light source is emitted along a line substantially parallel to the axis of the cone.

7. A device for determining the dominant eye of a person, the device comprising:
a) a cone having a hole at its apex which serves as a view port;
b) a handle attached to the cone at a hinge;
c) a light source; and
d) a light port disposed in the cone, handle or hinge to allow light from the light source to be emitted along a line substantially parallel to the axis of the cone.

8. A device for determining the dominant eye of a person, the device comprising:
a) a cone having a hole at its apex which serves as a view port;
b) a handle attached to the cone, the handle having a light port; and
c) a light assembly disposable in the handle to cause light to be emitted through the light port.

9. The device of claim 8 wherein the light assembly comprises:
a) a housing sized to fit within the handle;
b) a light source in the housing;
c) a battery connected to the light source; and
d) a switch connected to the light source.

10. The device of claim 8 wherein the housing further comprises a cap, wherein the cap has an outside diameter larger than the handle and an inside diameter larger than the handle, such that when the light assembly is inserted in the handle, the handle fits in the space between the inside surface of the cap and the outside surface of the housing cylinder.

11. The device of claim 10 wherein the switch is disposed on the cap.

12. The device of claim 8 wherein the housing further comprises a light port through which the light from the light source is emitted.

13. The device of claim 8 wherein the light from the light source is emitted along a line substantially parallel to the axis of the cone.

14. A manufacture for making a device for determining the dominant eye of a person, the manufacture comprising:
a) a single sheet of material comprising:
i. a cone-formative portion;
ii. a hinge-formative portion; and
iii. a handle-formative portion.

15. The manufacture of claim 14 wherein the cone-formative portion further comprises:
a) a first edge and a second edge that are joined together to form a cone.

16. The manufacture of claim 14 wherein the handle-formative portion further comprises:
a) a third edge and a fourth edge that are joined together to form a handle.

17. The manufacture of claim 16 further comprising:
a) a light assembly disposable in the handle, the light assembly comprising:
i. a light source;
ii. a battery in connection with the light source; and
iii. a switch in connection with the light source.

18. A device for determining the dominant eye of a person, the device comprising:
a) a cone made from a cone-formative portion of a single sheet of material, the cone having a hole at its apex which serves as a view port;
b) a handle made from a handle-formative portion of the single sheet of material, the handle attached to the cone, and the handle having a light port; and
c) a light source contained in the handle which emits light through the light port.

19. The device of claim 18 wherein the light from the light source is emitted along a line substantially parallel to the axis of the cone.

20. The device of claim 18 further comprising:
a) a battery in connection with the light source; and
b) a switch in connection with the light source.
